(19) United States
(12) Patent Application Publication
(10) Pub. No.: US 2018/0265914 A1
(43) Pub. Date: Sep. 20, 2018

(54) MOLECULAR METHODS FOR ASSESSING POST KIDNEY TRANSPLANT COMPLICATIONS

(71) Applicants: Hitachi Chemical Co., Ltd., Tokyo (JP); Hitachi Chemical Co. America, Ltd., San Jose, CA (US); City of Sapporo, Sapporo-shi, Hokkaido (JP)

(72) Inventors: Taku Murakami, Irvine, CA (US); Cindy M. Yamamoto, Irvine, CA (US); Masato Mitsuhashi, Irvine, CA (US); Hiroshi Harada, Sapporo (JP)

(21) Appl. No.: 15/756,488

(22) PCT Filed: Aug. 30, 2016
(86) PCT No.: PCT/US16/49473

§ 371 Date: Feb. 28, 2018

Related U.S. Application Data
(60) Provisional application No. 62/212,459, filed on Aug. 31, 2015.

(51) Int. Cl. C12Q 1/6809; C12Q 1/6876; C12Q 1/686; C12Q 1/6806; G01N 33/50; G01N 33/53; G01N 33/68

(52) U.S. Cl. CPC C12Q 1/6809; C12Q 1/6876; C12Q 1/686; C12Q 1/6806; G01N 33/5091; G01N 33/5308; G01N 2800/245; C12Q 2531/113; C12Q 2539/10; C12Q 2600/112; C12Q 2600/118; C12Q 2600/16; C12Q 2600/158; G01N 33/6893

(57) ABSTRACT

The present disclosure relates to methods of collecting exosomes and microvesicles (EMV) from urine, isolating corresponding mRNA, and analyzing expression patterns in order to diagnose and treat various post-kidney transplant complications. In particular, annexin1 mRNA expression patterns are analyzed through a unique diagnostic formula.

### INCORPORATION BY REFERENCE TO ANY PRIORITY APPLICATIONS

Any and all applications for which a foreign or domestic priority claim is identified in the Application Data Sheet as filed with the present application are hereby incorporated by reference and made part of the present disclosure.

### BACKGROUND

### Field

Several embodiments of the methods and systems disclosed herein relate to monitoring of a post-transplant kidney condition. Several embodiments relate to characterizing mRNA profiles of exosomes and microvesicles from urine samples to assess kidney condition.

### Description of the Related Art

Kidney transplantation is the last resort for end stage renal disease patients. Although more than 100,000 patients are waiting for kidney transplants (median wait time: 3.6 years), only about 17,000 kidney transplants took place in 2014 due to the limitation of donors and the gap between the patients and donors keeps growing. Development of immunosuppressant drugs improved graft survival significantly in recent years, however post-transplant complications including acute and chronic rejections are still the leading causes of graft loss followed by other complications.

Conventional urinary biomarkers such as serum creatinine, urinary creatinine and urine protein are not sensitive and specific enough to predict post-transplant complications so far. Kidney biopsy has been the gold standard to diagnose graft status and decide treatment strategies, however not an ideal solution for frequent monitoring due to its invasive nature and financial burdens to patients. Especially for patients receiving anti-platelet and anti-coagulant medicines due to for uremic platelet dysfunction, altered vessel architecture and other factors, kidney biopsy is not applicable or become risky. Therefore, non-invasive biomarkers for post-transplant kidney monitoring are desired.

### SUMMARY

There are provided herein, in several embodiments, methods and systems for identifying such biomarkers, and using such biomarkers to direct a specific treatment for a patient after kidney transplantation. In several embodiments, the methods are computer-based, and allow an essentially real-time determination of kidney status. In several embodiments, the methods lead to a determination of kidney status, while in some embodiments, a specific recommended treatment paradigm is produced (e.g., for a medical professional to act on).

In certain aspects, various RNA can be used in the methods, including, but not limited to detecting the presence of a post-kidney transplant complication in a subject. In several embodiments, the method includes detecting the levels of markers to successfully diagnose acute cellular rejection as well as to predict the rejections prior to an invasive biopsy (e.g., up to 20 days before a biopsy would confirm diagnosis). Samples used can include blood, urine, or any other biological sample. In certain variants, the method includes quantifying mRNA expression in exosomes and microvesicles isolated from a urine sample of the patient. In some embodiments, the method includes detecting the levels of ANXA1 in a urine sample from the subject, wherein ANXA1 is in urinary exosomes and microvesicles, and wherein the detection of an elevated level of at least one marker indicates the presence of post-kidney transplant complication in the subject. In some embodiments, the post-kidney transplant complication is selected from the group consisting of acute rejection, chronic rejection, borderline, interstitial fibrosis and tubular atrophy, immunoglobulin A (IgA) nephropathy and calcineurin inhibitor (CNI) toxicity. In certain variants, the method further includes a step to detect a reference gene selected from the group consisting of ACTB and GAPDH, wherein said reference gene is used to normalize a level of the at least one marker. In some embodiments, an elevated level is a level that is more than 2-fold increase compared to the level of a the marker in a urine sample of a donor without post-kidney transplant complications.

In some embodiments, a method is disclosed for screening a human subject for an expression of an RNA associated with a post-kidney transplant complication, the method comprising comparing an expression of the RNA in a vesicle isolated from a urine sample from the subject with an expression of the RNA in a vesicle isolated from a urine sample of a donor without post-kidney transplant complications, wherein the RNA associated with a post-kidney transplant complication is ANXA1, wherein an increase in said expression of the RNA of the subject compared to the expression of the RNA of the donor indicates the subject has a post-kidney transplant complication when the increase is beyond a threshold level, wherein the comparing the expression of the RNA in the vesicle isolated from the urine sample further comprises: capturing the vesicle from the sample from the subject by moving the sample from the subject across a vesicle-capturing filter, loading a lysis buffer onto the vesicle-capturing filter, thereby lysing the vesicle to release a vesicle-associated RNA, quantifying the expression of the RNA associated with a post-kidney transplant complication in the vesicle-associated RNA by PCR. In some variants, the method further includes using analytical software to determine a marker cycle threshold (Ct) value for the RNA associated with a post-kidney transplant complication, using analytical software to determine a reference Ct value for a reference RNA, and subtracting the marker Ct value from the reference Ct value to obtain a marker delta Ct value. In some variants, the reference RNA is selected from the group consisting of ACTB and GAPDH. In some embodiments, the increase is beyond the threshold level when the marker delta Ct value is less than 6. In some embodiments, the method includes comparing the marker delta Ct value to a control delta Ct value, the control delta Ct value being determined by subtracting a control marker Ct value from a control reference Ct value, the control marker Ct value being a Ct value of said RNA associated with a post-kidney transplant complication in urinary vesicles of a healthy donor population, the control reference Ct value being a Ct value of said reference RNA in urinary vesicles of a healthy donor population. In some embodiments, the increase is beyond the threshold level when the marker delta Ct value is at least 2 less than the control delta Ct value.

The methods summarized above and set forth in further detail below describe certain actions taken by a practitioner; however, it should be understood that they can also include the instruction of those actions by another party. Thus, actions such as “treating a subject for a disease or condition” include “instructing the administration of treatment of a subject for a disease or condition.”

### BRIEF DESCRIPTION OF THE DRAWINGS

Various embodiments are depicted in the accompanying drawings for illustrative purposes, and should in no way be interpreted as limiting the scope of the embodiments. Furthermore, various features of different disclosed embodiments can be combined to form additional embodiments, which are part of this disclosure.

FIG. 1A shows a plot of Annexin A1 (ANXA1) mRNA expression in a subject's urinary EMV as a function of that subject's Banff score for interstitial fibrosis (ci).

FIG. 1B shows a plot of ANXA1 mRNA expression in a subject's urinary EMV as a function of that subject's Banff score for tubular atrophy (ct).

FIG. 1C shows a plot of ANXA1 mRNA expression in a subject's urinary EMV as a function of that subject's Banff score for total interstitial inflammation (ti).

FIG. 1D shows a plot of ANXA1 mRNA expression in a subject's urinary EMV as a function of that subject's Banff score for tubulitis (t).

FIG. 1E shows a plot of ANXA1 mRNA expression in a subject's urinary EMV as a function of that subject's Banff score for interstitial infiltration (i).

FIG. 1F shows a plot of ANXA1 mRNA expression in a subject's urinary EMV as a function of that subject's Banff score for intimal arteritis (v).

FIG. 1G shows a plot of ANXA1 mRNA expression in a subject's urinary EMV as a function of that subject's Banff score for hyaline arteriolar thickening (ah).

FIG. 1H shows a plot of ANXA1 mRNA expression in a subject's urinary EMV as a function of that subject's Banff score for tubular peritubular capillaritis (ptc).

FIG. 1I shows a plot of ANXA1 mRNA expression in a subject's urinary EMV as a function of that subject's Banff score for glomerultitis (g).

FIG. 1J shows a plot of ANXA1 mRNA expression in a subject's urinary EMV as a function of that subject's Banff score for vascular fibrosis intimal thickening (cv).

FIG. 1K shows a plot of ANXA1 mRNA expression in a subject's urinary EMV as a function of that subject's Banff score for alternative scoring of hyaline arteriolar thickening (aah).

FIG. 1L shows a plot of ANXA1 mRNA expression in a subject's urinary EMV as a function of that subject's Banff score for peritubular capillaritis as determined by Banff Method (ptcbm).

FIG. 1M shows a plot of ANXA1 mRNA expression in a subject's urinary EMV as a function of that subject's Banff score for chronic glomerulopathy (cg).

FIG. 1N shows a plot of ANXA1 mRNA expression in a subject's urinary EMV as a function of that subject's Banff score for complement C4d staining (c4d).

FIG. 1O shows a plot of ANXA1 mRNA expression in a subject's urinary EMV as a function of that subject's Banff score for mesangial matrix increase (mm).

FIG. 2A shows a scatter plot of ANAX1 mRNA expression in a subject's urinary EMV as a function of that subject's urine protein level.

FIG. 2B shows a scatter plot of ANAX1 mRNA expression in a subject's urinary EMV as a function of that subject's urine creatinine level.

FIG. 2C shows a scatter plot of ANAX1 mRNA expression in a subject's urinary EMV as a function of that subject's serum creatinine level.

FIG. 2D shows a scatter plot of ANAX1 mRNA expression in a subject's urinary EMV as a function of that subject's estimated glomerular filtration rate.

FIG. 3 shows a schematic diagram of patient and sample classification.

FIG. 4 shows a plot of ANXA1 mRNA expression in a subject's urinary EMV as a function of that subject's status regarding various types of post-kidney transplant conditions. Statistical significance was determined by Mann-Whitney-Wilcoxon test: one star (*) for p<0.05 and four (****) for p<0.0001.

FIG. 5A shows a plot of ANXA1 mRNA expression in a subject's urinary EMV as a function of time from the date that transplant rejection is confirmed in that subject. Statistical significance was determined by Mann-Whitney-Wilcoxon test: two stars (**) for p<0.01, three (***) for p<0.001 and four (****) for p<0.0001.

FIG. 5B shows Receiver Operating Characteristic (ROC) analysis for ANXA1 mRNA expression before (solid line), during (perforated line) and after (dotted line) the confirmation of transplant rejection.

FIG. 5C shows a plot of ANXA1 mRNA expression in a subject's urinary EMV as a function of time from the date that interstitial fibrosis and tubular atrophy (IFTA) is confirmed in that subject. Statistical significance was determined by Mann-Whitney-Wilcoxon test: two stars (**) for p<0.01, three (***) for p<0.001 and four (****) for p<0.0001.

FIG. 5D shows Receiver Operating Characteristic (ROC) analysis for ANXA1 mRNA expression before (solid line), during (perforated line) and after (dotted line) the confirmation of IFTA.

FIG. 5E shows a plot of ANXA1 mRNA expression in a subject's urinary EMV as a function of time from the date that other complication such as Immunoglobulin A (IgA) nephropathy and calcineurin inhibitor (CNI) toxicity is confirmed in that subject. Statistical significance was determined by Mann-Whitney-Wilcoxon test: two stars (**) for p<0.01, three (***) for p<0.001 and four (****) for p<0.0001.

FIG. 5F shows Receiver Operating Characteristic (ROC) analysis for ANXA1 mRNA expression before (solid line), during (perforated line) and after (dotted line) the confirmation of complications.

### DETAILED DESCRIPTION

Certain aspects of the present disclosure are generally directed to a minimally-invasive method that monitors a patient's post-kidney transplant condition. Each and every feature described herein, and each and every combination of two or more of such features, is included within the scope of the present disclosure provided that the features included in such a combination are not mutually inconsistent.

Exosomes and microvesicles (EMV) are released into the urinary space from all the areas of the nephrons by encapsulating the cytoplasmic molecules of the cell of origin. EMV are considered a promising source of biomarkers as urinary EMV mRNA profiles reflect kidney functions and injuries. Compared to conventional non-invasive biomarker sources such as urine cells and blood, urinary EMV may contain earlier and clearer signatures of kidney injuries. Messenger RNA (mRNA) expression of CD3E and CXCL10 (e.g., compared to 18S rRNA levels) has been measured in cells collected from urine but has not been reported for urinary EMV. Also, mRNA expression of additional markers (e.g., CFLAR, DUSP1, IFNGR1, ITGAX, MAPK9, NAMPT, NKTR, PSEN1, RNF130, RYBP, CEACAM4, EPOR, GZMK, RARA, RHEB, RXRA, SLC25A37, and the like) was measured in whole blood but not urinary EMV. Monitoring of post-transplant kidney condition is important for the management of long term graft survival. Urinary cells are released into urine after severe injuries of the nephrons, however urinary EMV are released not only from injured cells but also from normal cells. Therefore, injury related molecular signatures could be obtained from the injured cells before the injured cells are released into urine. Thus, several embodiments of the present disclosure take advantage of EMVs from urine.

The standard method to isolate urinary EMV is a differential centrifugation method using ultracentrifugation. However, use of ultracentrifugation may not be applicable for routine clinical assays at regular clinical laboratories. Several embodiments of the present disclosure employ a urinary EMV mRNA assay for biomarker and clinical studies, which enables similar or even superior performances to the standard method in terms of assay sensitivity, reproducibility and ease of use. Several embodiments employ this urinary EMV mRNA assay to screen kidney injury markers for post-transplant graft monitoring, advantageously at time periods well in advance of those utilizing standard diagnostic techniques (e.g., biopsy).

As described in more detail below, urinary exosomes can be isolated from urine by passing urine samples through a vesicle capture filter, thereby allowing the EMV to be isolated from urine without the use of ultracentrifugation. In some embodiments, the vesicle capture material has a porosity that is orders of magnitude larger than the size of the captured vesicle. Although the vesicle-capture material has a pore size that is much greater than the size of the EMV, the EMV are captured on the vesicle-capture material by adsorption of the EMV to the vesicle-capture material. The pore size and structure of the vesicle-capture material is tailored to balance EMV capture with EMV recovery so that mRNA from the EMV can be recovered from the vesicle-capture material. In some embodiments, the vesicle-capture material is a multi-layered filter that includes at least two layers having different porosities. In one embodiment, the first layer has a particle retention rate between 0.6 and 2.7 μm, preferably 1.5 and 1.8 μm, and the second layer has a particle retention rate between 0.1 and 1.6 μm, preferably 0.6 and 0.8 μm. In one embodiment, a particle retention rate of the first layer is greater than that of the second layer, thereby higher particulate loading capacity and faster flow rates can be obtained. In some embodiments, the urine sample passes first through a first layer and then through a second layer, both made of glass fiber. The first layer has a pore-size of 1.6 μm, and the second layer has a pore size of 0.7 μm.

In several embodiments, the methods of the present disclosure use a filter-based EMV mRNA assay to screen urine samples of post-kidney transplant patients to monitor kidney condition. In some embodiments, the methods disclosed herein can be used to screen EMV mRNA that is obtained from vesicles which have been isolated from urine by ultracentrifugation. In several embodiments, urinary EMV are screened for kidney injury biomarkers that can be detected before kidney injury can be detected by the current standard practice of evaluating transplant rejection (e.g., kidney biopsy).

Peripheral blood is a rich source of biomarkers for many diseases and organ damages. However, injury related signatures from kidney may be diluted and mixed with EMV released into the peripheral blood from other organs. Urinary EMV mRNAs have been shown to predict post-transplant outcomes in some circumstances. However, certain genes studied, such as LCN2 (NGAL), IL18, HAVCR1 (KIM1) and CST3 (cystatin C), were not routinely correlated with urinary protein biomarkers or with day 7 creatinine reduction ratios. Several embodiments of the methods and systems disclosed herein relate to monitoring of post-transplant kidney condition, which is important for the management of long term graft survival.

Several aspects of the present disclosure employ a urinary exosome and microvesicle (EMV) mRNA assay in which early kidney injury biomarkers are screened from patients who received kidney transplantation. Various mRNA are informative regarding the status of a post-transplant kidney, including, but not limited to Annexin A1 (ANXA1). As discussed below, several embodiments of the methods herein disclosed indicate that ANXA1 expression level is linearly correlated with Banff scores ci (interstitial fibrosis), ct (tubular atrophy) and ti (total interstitial inflammation) of the matched kidney biopsies (N=117). Compared to the patients with stable recovery, annexin A1 (ANXA1) expression in urinary EMV increased when T-cell mediated rejection (TCMR, 10.2-fold), antibody mediated rejection (ABMR, 14.4-fold), interstitial fibrosis and tubular atrophy (IFTA, 22.0-fold) and other complications (8.7-fold) were observed. ANXA1 increased at least 6.5 days before transplant rejection, 56 days before IFTA and 64 days before other complications, and remained high after the complications disappeared. ROC curve analysis indicated that urinary ANXA1 was able to predict and diagnose post-transplant complications accurately: transplant rejection (AUC=0.857 to 0.946), IFTA (AUC=0.777 to 0.995) and other complications (AUC=0.698 to 0.797). Thus, in accordance with several embodiments disclosed herein, the methods and systems employing urinary EMV ANXA1 mRNA analysis are effective at early predictions of interstitial fibrosis and tubular atrophy and useful for post-transplant graft monitoring.

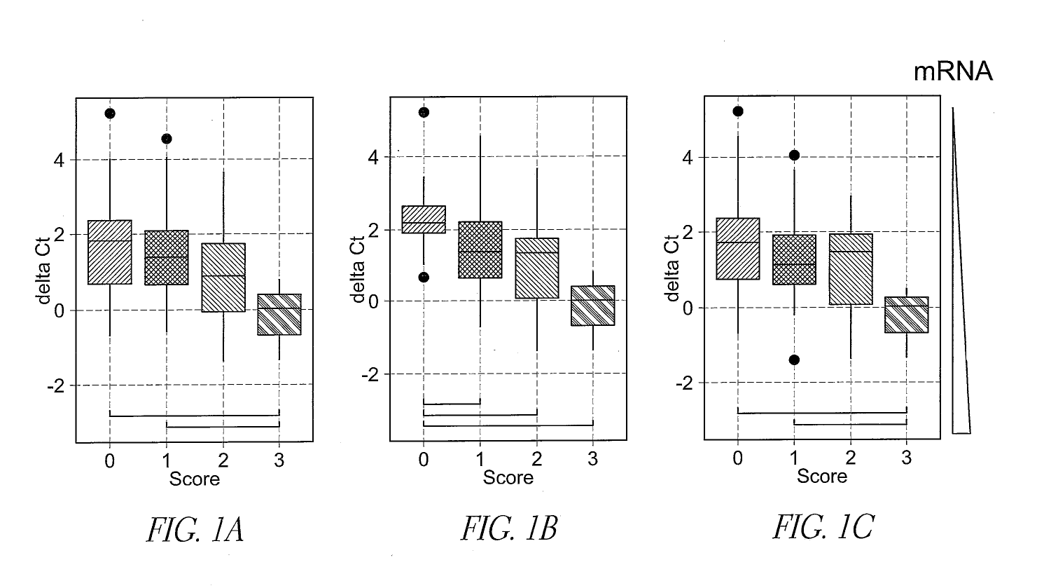

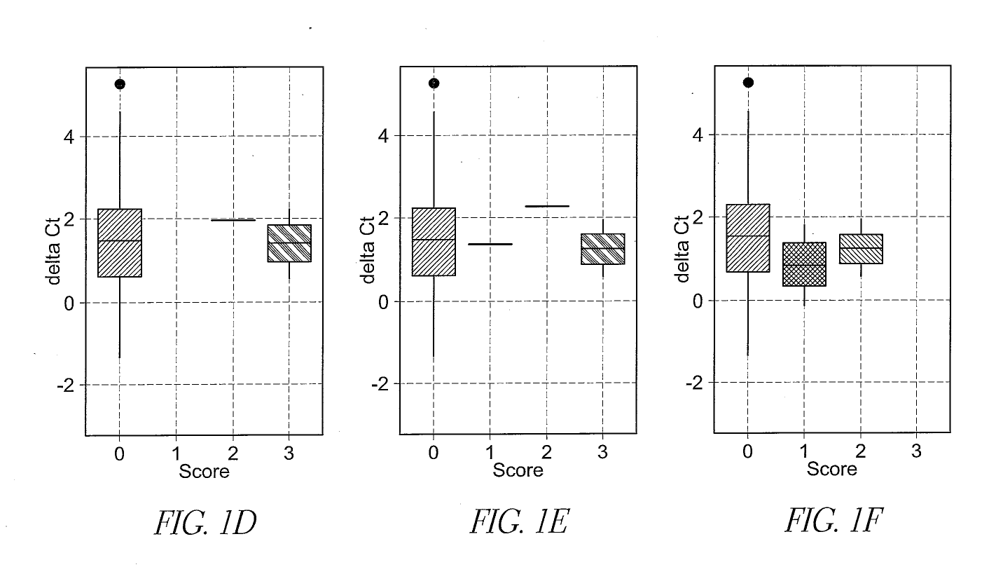

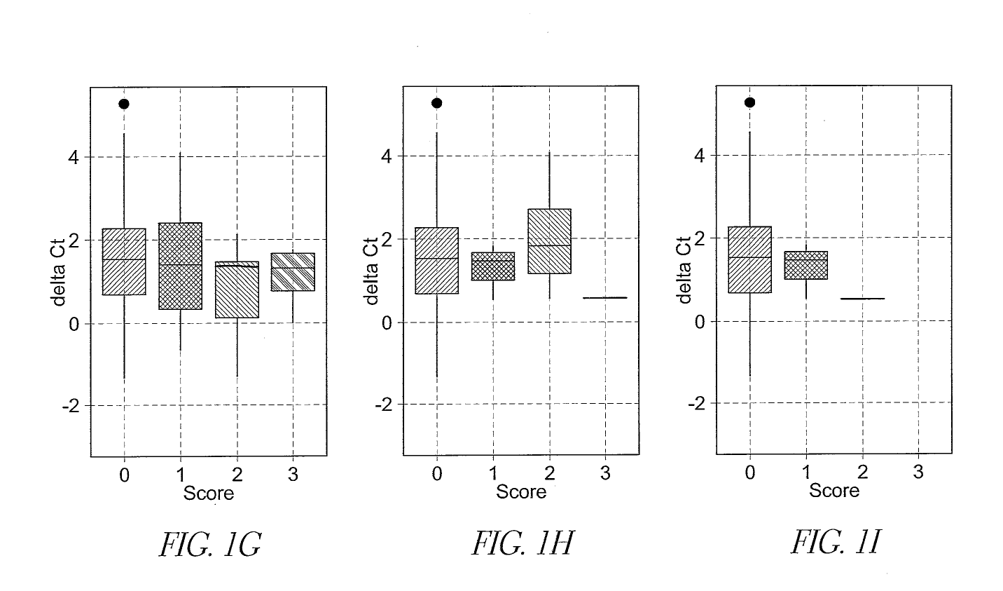

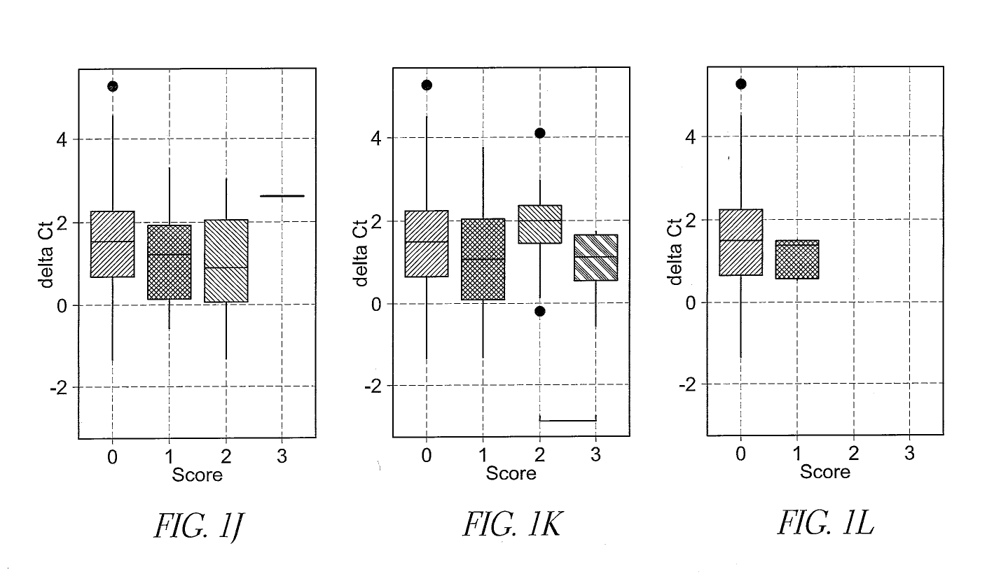

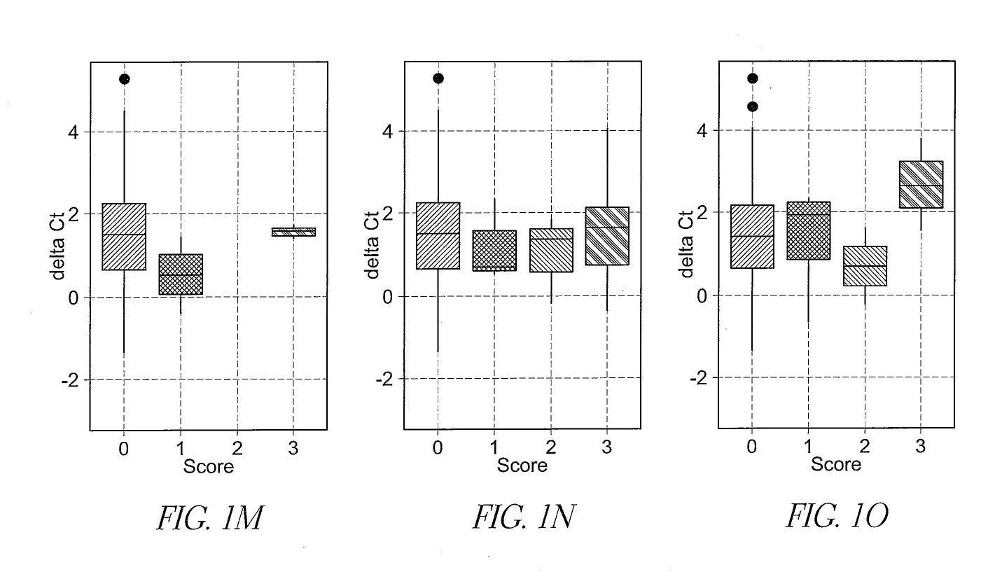

Urinary EMV ANXA1 mRNA expression levels were compared to the matched biopsy scores of post-kidney transplant patients. As shown in FIGS. 1A-C, urinary EMV ANXA1 expression level was linearly correlated with Banff scores for interstitial fibrosis (“ci”, FIG. 1A), tubular atrophy (“ct”, FIG. 1B), and total interstitial inflammation (“ti”, FIG. 1C). As shown in FIGS. 1D-O, urinary EMV ANXA1 expression level was not correlated with Banff scores for tubulitis (“t”, FIGURE D), interstitial infiltration (“i”, FIG. 1E), intimal arteritis (“v”, FIG. 1F), hyaline arteriolar thickening (“ah”, FIG. 1G), tubular peritubular capillaritis (“ptc”, FIG. 1H), glomerultitis (“g”, FIG. 1I), vascular fibrosis intimal thickening (“cv”, FIG. 1J), alternative scoring for hyaline arteriolar thickening (“aah”, FIG. 1K), peritubular capillaritis as determined by Banff Method (“ptcbm”, FIG. 1L), chronic glomerulopathy (“cg”, FIG. 1M), complement C4d staining (“c4d”, FIG. 1N), and mesangial matrix increase (“mm”, FIG. 1O). Accordingly, ANXA1 mRNA in EMV may be a promising biomarker indicating interstitial fibrosis and tubular atrophy.

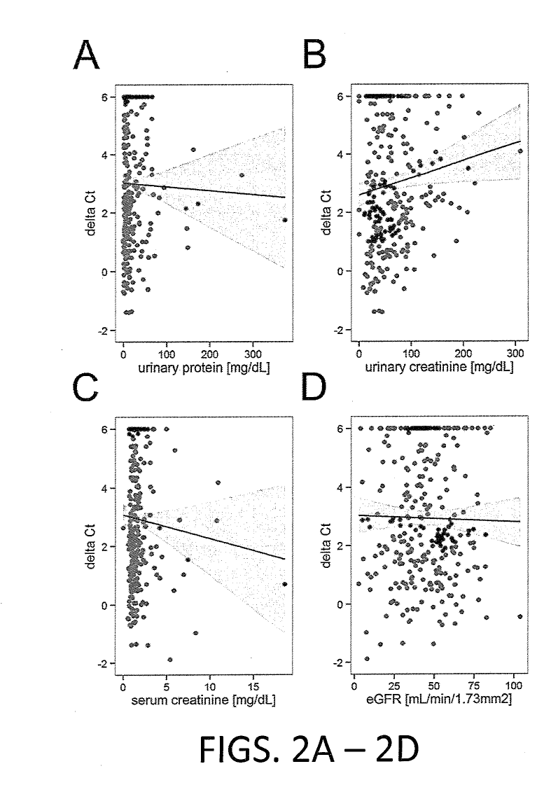

FIGS. 2A-D show that urinary EMV ANXA1 mRNA expression level does not display any correlation with conventional markers of post-kidney transplant complication and/or rejection. Urinary EMV ANXA1 mRNA expression did not show any association with urine protein concentration (FIG. 2A), urinary creatinine concentration (FIG. 2B), serum creatinine concentration (FIG. 2C), and estimated glomerular filtration rate (FIG. 2D).

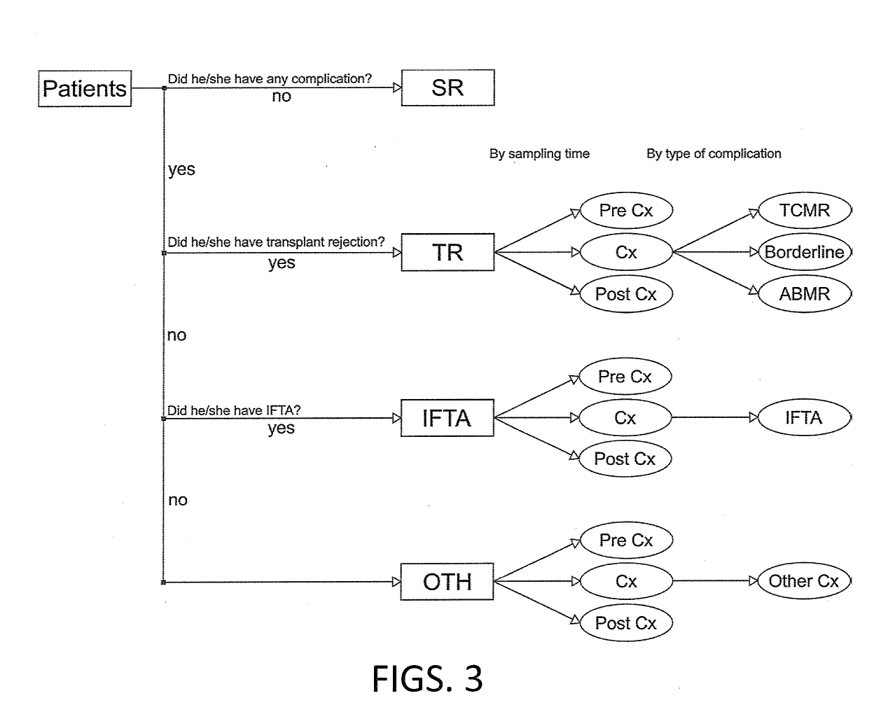

ANXA1 mRNA expression in urinary EMV was evaluated for patients with various types of post-transplant complications and for patients with stable post-operative recovery during the study period. Post-transplant patients were categorized into four groups by the complications that the patients were diagnosed with during the study period: stable recovery (SR), transplant rejection (TR), interstitial fibrosis and tubular atrophy (IFTA) and other complications (OTH) (FIG. 3, Table 1). For the TR, IFTA and OTH patient groups, urine samples were categorized into three groups by sampling time relative to the time when complications were observed: Pre Cx, Cx and Post Cx (FIG. 3, Table 2). Urine samples collected when the TR and IFTA patients showed other complications such as Immunoglobulin A (IgA) nephropathy and calcineurin inhibitor (CNI) toxicity were also categorized as Cx. Pre Cx samples were the samples collected before the first complication observed during the study period, and Post Cx were after the last one. It should be noted that relative sampling time of Pre Cx in the TR group was median 6.5 (IQR 5-9) days before the first complication and skewed compared with those of the IFTA (median 56 (IQR 43-168) days before) and OTH (median 64 (IQR 27-177) days before). The Cx samples were further categorized by the type of complications observed during sample collection: TCMR, Borderline, ABMR, IFTA and other complications (FIG. 3).

TABLE 1
Patient categories showing for each sample category
median and IQR values of post-operation day (POD).

| Patient group | Subject | Sample | Median POD (IQR) | |
|---|---|---|---|---|
| Stable recovery (SR) | 34 | 52 | 240.5 | (24-793) |
| Transplant rejection (TR) | 20 | 50 | 364 | (52-737) |
| Interstitial fibrosis and | 51 | 98 | 365 | (21-1036) |
| tubular atrophy (IFTA) | | | | |
| Other complications (OTH) | 50 | 99 | 94.5 | (11-906) |
| Total | 155 | 299 | 240.5 | (14-901) |

TABLE 2
Sample categories showing for each sample category
median and IQR values of sampling day relative
to the time complication was observed

| | | Median relative | | |
|---|---|---|---|---|
| Patient group | Sample group | sampling day (IQR) | | Sample |
| TR | Pre Cx | −6.5 | (−9 to −5) | 6 |
| | Cx | 0 | | 30 |
| | Post Cx | +288.5 | (+223 to +347) | 8 |
| IFTA | Pre Cx | −56 | (−168 to −43) | 33 |
| | Cx | 0 | | 59 |
| | Post Cx | +176.5 | (+54 to +319) | 4 |
| OTH | Pre Cx | −64 | (−177 to −27) | 39 |
| | Cx | 0 | | 50 |
| | Post Cx | +58 | (+38 to +75) | 9 |

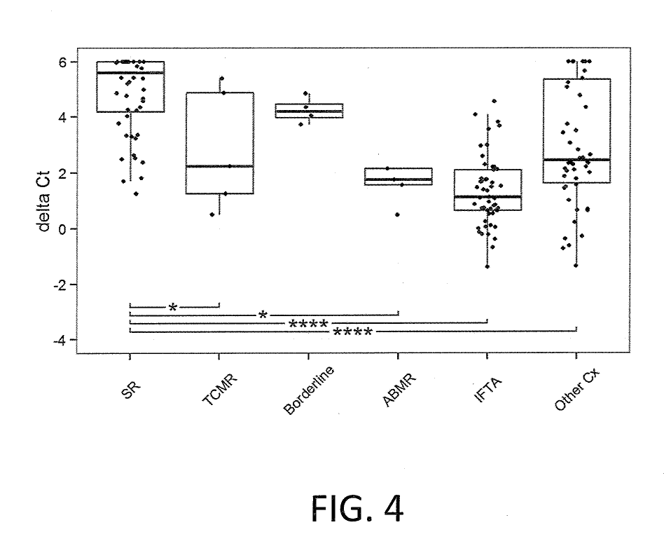

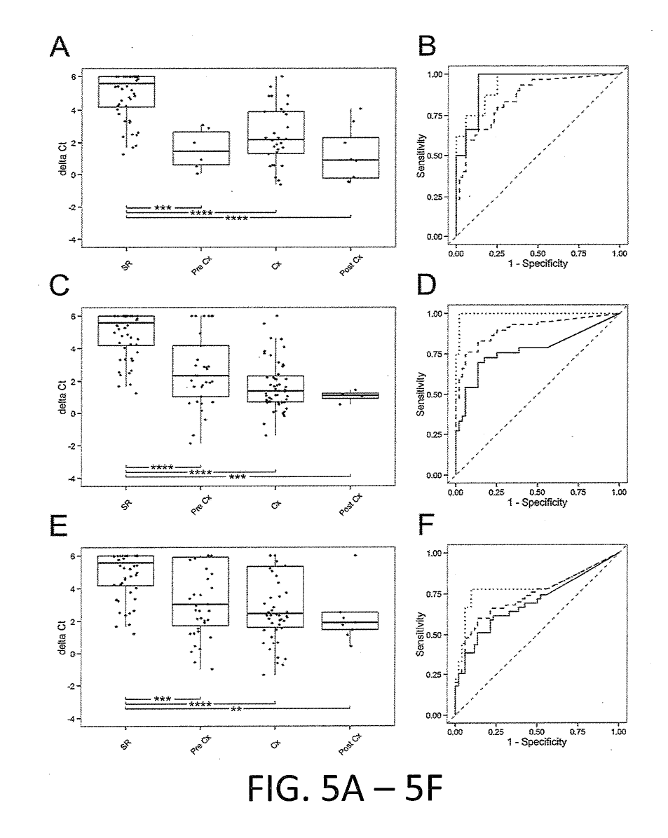

FIG. 4 shows urinary EMV ANXA1 mRNA expression levels in stable recovery patients and in patients displaying post-transplant complications. Expression level of ANXA1 in urinary EMV was analyzed in the samples obtained when post-transplant complications were observed in comparison with those of the SR group (FIG. 4). Increase of ANXA1 level was observed in TCMR (10.2-fold increase, p=0.017), ABMR (14.4-fold increase, p=0.015), IFTA (22.0-fold increase, p=4.3×10⁻¹⁶) and other complications (8.7-fold increase, p=2.2×10⁻⁵). On the other hand, ANXA1 increased by at least 2.6-fold in Borderline, however statistical significance was not observed.

To evaluate predictive and prognostic values of ANXA1 in post-transplant graft monitoring, the urine samples in the TR, IFTA and OTH patients were categorized by sampling time and analyzed. Compared to the SR patients, the TR patients showed an increase of ANXA1 level at least a median of 6.5 (IQR 5 to 9) days before the first complication was observed and remained high for a median of 288.5 (IQR 222.5 to 346.8) days after the last complication (FIG. 4A). ROC curve analysis showed that the expression level of ANXA1 can distinguish the TR patients from the SR with AUC=0.946 (Pre Cx), 0.857 (Cx), and 0.940 (Post Cx) (FIG. 4B, Table 3).

The IFTA and OTH patients also showed increase of ANXA1 independent of sampling time, just like the TR patients. However, the increase was observed much earlier or at least for a median of 56 (IQR 43 to 168) and 64 (IQR 27 to 177) days before the first complication, respectively (FIG. 4C, 4E). ROC curve analysis indicated that ANXA1 can distinguish the IFTA patients from the SR patients with comparable sensitivity and specificity to the TR patients: AUC 0.777 (Pre Cx), 0.906 (Cx), and 0.995 (Post Cx) (FIG. 4D, Table 3). On the other hand, the OTH patients were less sensitive and specific compared to other complication groups but still the obtained AUCs were 0.698 to 0.797 (FIG. 3E, Table 3).

TABLE 3
Diagnostic performance of urinary EMV ANXA1.

| | Pre Cx | Cx | Post Cx |
|---|---|---|---|
| Patient group | (AUC) | (AUC) | (AUC) |
| TR | 0.946 | 0.857 | 0.940 |
| IFTA | 0.777 | 0.906 | 0.995 |
| OTH | 0.698 | 0.739 | 0.797 |

In some embodiments of the present disclosure, up-regulation of ANXA1 can indicate the need for biopsy confirmation of the kidney condition. Although ANXA1 did not distinguish between post-transplant complications, elevated levels of urinary EMV ANXA1 mRNA can predict graft rejection at least 6.5 days earlier than the current practice and can predict IFTA and other complications at least 56 and 64 days earlier, respectively. Given the injurious and invasive nature of biopsy, the methods of the present disclosure can assist early treatment of post-kidney transplant complications by limiting the use of biopsy to situations when a biopsy is indicated by elevated levels of ANXA1 mRNA expression in urinary EMV.

As discussed above, there are provided herein several embodiments in which nucleic acids are evaluated from blood or urine samples in order to detect and determine an expression level of a particular marker. In several embodiments, the determination of the expression of the marker allows a diagnosis of a disease or condition, for example kidney injury. In several embodiments, the determination is used to measure the severity of the condition and develop and implement an appropriate treatment plan. In several embodiments, the detected biomarker is then used to develop an appropriate treatment regimen. In several embodiments, however, the treatment may be taking no further action (e.g., not instituting a treatment). In several embodiments the methods are computerized (e.g., one or more of the RNA isolation, cDNA generation, or amplification are controlled, in whole or in part, by a computer). In several embodiments, the detection of the biomarker is real time.

As above, certain aspects of the methods are optionally computerized. Also, in several embodiments, the amount of expression may result in a determination that no treatment is to be undertaken at that time. Thus, in several embodiments, the methods disclosed herein also reduce unnecessary medical expenses and reduce the likelihood of adverse effects from a treatment that is not needed at that time.

In some embodiments, after a biological sample is collected (e.g., a urine sample), membrane particles, cells, exosomes, exosome-like vesicles, microvesicles and/or other biological components of interest are isolated by filtering the sample. In some embodiments, filtering the collected sample will trap one or more of membrane particles, exosomes, exosome-like vesicles, and microvesicles on a filter. In some embodiments, the vesicle-capturing material captures desired vesicles from a biological sample. In some embodiments, therefore, the vesicle-capturing material is selected based on the pore (or other passages through a vesicle-capturing material) size of the material. In some embodiments, the vesicle-capturing material comprises a filter.

In some embodiments, the filter comprises pores. As used herein, the terms “pore” or “pores” shall be given their ordinary meaning and shall also refer to direct or convoluted passageways through a vesicle-capture material. In some embodiments, the materials that make up the filter provide indirect passageways through the filter. For example, in some embodiments, the vesicle-capture material comprises a plurality of fibers, which allow passage of certain substances through the gaps in the fiber, but do not have pores per se. For instance, a glass fiber filter can have a mesh-like structure that is configured to retain particles that have a size of about 1.6 microns or greater in diameter. Such a glass fiber filter may be referred to herein interchangeably as having a pore size of 1.6 microns or as comprising material to capture components that are about 1.6 microns or greater in diameter. However, as discussed above, the EMV that are captured by the filter are orders of magnitude smaller than the pore size of the glass filter. Thus, although the filter may be described herein as comprising material to capture components that are about 1.6 microns or greater in diameter, such a filter may capture components (e.g., EMV) that have a smaller diameter because these small components may adsorb to the filter.

In some embodiments, the filter comprises material to capture components that are about 1.6 microns or greater in diameter. In several embodiments, a plurality of filters are used to capture vesicles within a particularly preferred range of sizes (e.g., diameters). For example, in several embodiments, filters are used to capture vesicles having a diameter of from about 0.2 microns to about 1.6 microns in diameter, including about 0.2 microns to about 0.4 microns, about 0.4 microns to about 0.6 microns, about 0.6 microns to about 0.8 microns, about 0.8 microns to about 1.0 microns, about 1.0 microns to about 1.2 microns, about 1.2 to about 1.4 microns, about 1.4 microns to about 1.6 microns (and any size in between those listed). In other embodiments, the vesicle-capture material captures exosomes ranging in size from about 0.5 microns to about 1.0 microns.

In some embodiments, the filter (or filters) comprises glass-like material, non-glass-like material, or a combination thereof. In some embodiments, wherein the vesicle-capture material comprises glass-like materials, the vesicle-capture material has a structure that is disordered or “amorphous” at the atomic scale, like plastic or glass. Glass-like materials include, but are not limited to glass beads or fibers, silica beads (or other configuration), nitrocellulose, nylon, polyvinylidene fluoride (PVDF) or other similar polymers, metal or nano-metal fibers, polystyrene, ethylene vinyl acetate or other co-polymers, natural fibers (e.g., silk), alginate fiber, or combinations thereof. In certain embodiments, the vesicle-capture material optionally comprises a plurality of layers of vesicle-capture material. In other embodiments, the vesicle-capture material further comprises nitrocellulose.

In some embodiments, a filter device is used to isolate biological components of interest. In some embodiments, the device comprises: a first body having an inlet, an outlet, and an interior volume between the inlet and the outlet; a second body having an inlet, an outlet, an interior volume between the inlet and the outlet, a filter material positioned within the interior volume of the second body and in fluid communication with the first body; and a receiving vessel having an inlet, a closed end opposite the inlet and interior cavity. In some embodiments, the first body and the second body are reversibly connected by an interaction of the inlet of the second body with the outlet of the first body. In some embodiments, the interior cavity of the receiving vessel is dimensioned to reversibly enclose both the first and the second body and to receive the collected sample after it is passed from the interior volume of the first body, through the filter material, through the interior cavity of the second body and out of the outlet of the second body. In some embodiments, the isolating step comprises placing at least a portion of the collected sample in such a device, and applying a force to the device to cause the collected sample to pass through the device to the receiving vessel and capture the biological component of interest. In some embodiments, applying the force comprises centrifugation of the device. In other embodiments, applying the force comprises application of positive pressure to the device. In other embodiments, applying the force comprises application of vacuum pressure to the device. Examples of such filter devices are disclosed in PCT Publication WO 2014/182330 and PCT Publication WO 2015/050891, hereby incorporated by reference herein.

In some embodiments, the collected sample is passed through multiple filters to isolate the biological component of interest. In other embodiments, isolating biological components comprises diluting the collected sample. In other embodiments, centrifugation may be used to isolate the biological components of interest. In some embodiments, multiple isolation techniques may be employed (e.g., combinations of filtration selection and/or density centrifugation). In some embodiments, the collected sample is separated into one or more samples after the isolating step.

In some embodiments, RNA is liberated from the biological component of interest for measurement. In some embodiments, liberating the RNA from the biological component of interest comprises lysing the membrane particles, exosomes, exosome-like vesicles, and/or microvesicles with a lysis buffer. In other embodiments, centrifugation may be employed. In some embodiments, the liberating is performed while the membrane particles, exosomes, exosome-like vesicles, microvesicles and/or other components of interest are immobilized on a filter. In some embodiments, the membrane particles, exosomes, exosome-like vesicles, microvesicles and/or other components of interest are isolated or otherwise separated from other components of the collected sample (and/or from one another—e.g., vesicles separated from exosomes).

According to various embodiments, various methods to quantify RNA are used, including Northern blot analysis, RNase protection assay, PCR, RT-PCR, real-time RT-PCR, other quantitative PCR techniques, RNA sequencing, nucleic acid sequence-based amplification, branched-DNA amplification, mass spectrometry, CHIP-sequencing, DNA or RNA microarray analysis and/or other hybridization microarrays. In some of these embodiments or alternative embodiments, after amplified DNA is generated, it is exposed to a probe complementary to a portion of a biomarker of interest.

In some embodiments, a computerized method is used to complete one or more of the steps. In some embodiments, the computerized method comprises exposing a reaction mixture comprising isolated RNA and/or prepared cDNA, a polymerase and gene-specific primers to a thermal cycle. In some embodiments, the thermal cycle is generated by a computer configured to control the temperature time, and cycle number to which the reaction mixture is exposed. In other embodiments, the computer controls only the time or only the temperature for the reaction mixture and an individual controls on or more additional variables. In some embodiments, a computer is used that is configured to receive data from the detecting step and to implement a program that detects the number of thermal cycles required for the biomarker to reach a pre-defined amplification threshold in order to identify whether a subject is suffering from kidney injury or displaying kidney transplant rejection. In still additional embodiments, the entire testing and detection process is automated.

For example, in some embodiments, RNA is isolated by a fully automated method, e.g., methods controlled by a computer processor and associated automated machinery. In one embodiment a biological sample, such as a urine sample, is collected and loaded into a receiving vessel that is placed into a sample processing unit. A user enters information into a data input receiver, such information related to sample identity, the sample quantity, and/or specific patient characteristics. In several embodiments, the user employs a graphical user interface to enter the data. In other embodiments, the data input is automated (e.g., input by bar code, QR code, or other graphical identifier). The user can then implement an RNA isolation protocol, for which the computer is configured to access an algorithm and perform associated functions to process the sample in order to isolate biological components, such as vesicles, and subsequently processed the vesicles to liberate RNA. In further embodiments, the computer implemented program can quantify the amount of RNA isolated and/or evaluate and purity. In such embodiments, should the quantity and/or purity surpass a minimum threshold, the RNA can be further processed, in an automated fashion, to generate complementary DNA (cDNA). cDNA can then be generated using established methods, such as for example, binding of a poly-A RNA tail to an oligo dT molecule and subsequent extension using an RNA polymerase. In other embodiments, if the quantity and/or purity fail to surpass a minimum threshold, the computer implemented program can prompt a user to provide additional biological sample(s).

Depending on the embodiment, the cDNA can be divided into individual subsamples, some being stored for later analysis and some being analyzed immediately. Analysis, in some embodiments comprises mixing a known quantity of the cDNA with a salt-based buffer, a DNA polymerase, and at least one gene specific primer to generate a reaction mixture. The cDNA can then be amplified using a predetermined thermal cycle program that the computer system is configured to implement. This thermal cycle, could optionally be controlled manually as well. After amplification (e.g., real-time PCR), the computer system can assess the number of cycles required for a gene of interest (e.g. a marker of kidney injury or kidney transplant rejection) to surpass a particular threshold of expression. A data analysis processor can then use this assessment to calculate the amount of the gene of interest present in the original sample, and by comparison either to a different patient sample, a known control, or a combination thereof, expression level of the gene of interest can be calculated. A data output processor can provide this information, either electronically in another acceptable format, to a test facility and/or directly to a medical care provider. Based on this determination, the medical care provider can then determine if and how to treat a particular patient based on determining the presence of kidney injury or kidney transplant rejection. In several embodiments, the expression data is generated in real time, and optionally conveyed to the medical care provider (or other recipient) in real time.

In several embodiments, a fully or partially automated method enables faster sample processing and analysis than manual testing methods. In certain embodiments, machines or testing devices may be portable and/or mobile such that a physician or laboratory technician may complete testing outside of a normal hospital or laboratory setting. In some embodiments, a portable assay device may be compatible with a portable device comprising a computer such as a cell phone or lap top that can be used to input the assay parameters to the assay device and/or receive the raw results of a completed test from the assay device for further processing. In some embodiments, a patient or other user may be able to use an assay device via a computer interface without the assistance of a laboratory technician or doctor. In these cases, the patient would have the option of performing the test “at-home.” In certain of these embodiments, a computer with specialized software or programming may guide a patient to properly place a sample in the assay device and input data and information relating to the sample in the computer before ordering the tests to run. After all the tests have been completed, the computer software may automatically calculate the test results based on the raw data received from the assay device. The computer may calculate additional data by processing the results and, in some embodiments, by comparing the results to control information from a stored library of data or other sources via the internet or other means that supply the computer with additional information. The computer may then display an output to the patient (and/or the medical care provider, and/or a test facility) based on those results.

In some embodiments, a medical professional may be in need of genetic testing in order to diagnose, monitor and/or treat a patient. Thus, in several embodiments, a medical professional may order a test and use the results in making a diagnosis or treatment plan for a patient. For example, in some embodiments a medical professional may collect a sample from a patient or have the patient otherwise provide a sample (or samples) for testing. The medical professional may then send the sample to a laboratory or other third party capable of processing and testing the sample. Alternatively, the medical professional may perform some or all of the processing and testing of the sample himself/herself (e.g., in house). Testing may provide quantitative and/or qualitative information about the sample, including data related to the presence of a urothelial disease. Once this information is collected, in some embodiments the information may be compared to control information (e.g., to a baseline or normal population) to determine whether the test results demonstrate a difference between the patient's sample and the control. After the information is compared and analyzed, it is returned to the medical professional for additional analysis. Alternatively, the raw data collected from the tests may be returned to the medical professional so that the medical professional or other hospital staff can perform any applicable comparisons and analyses. Based on the results of the tests and the medical professional's analysis, the medical professional may decide how to treat or diagnose the patient (or optionally refrain from treating).

In several embodiments, filtration (alone or in combination with centrifugation) is used to capture vesicles of different sizes. In some embodiments, differential capture of vesicles is made based on the surface expression of protein markers. For example, a filter may be designed to be reactive to a specific surface marker (e.g., filter coupled to an antibody) or specific types of vesicles or vesicles of different origin. In several embodiments, the combination of filtration and centrifugation allows a higher yield or improved purity of vesicles.

In some embodiments, the markers are unique vesicle proteins or peptides. In some embodiments, the severity of a particular gynecological disease or disorder is associated with certain vesicle modifications which can be exploited to allow isolation of particular vesicles. Modification may include, but is not limited to addition of lipids, carbohydrates, and other molecules such as acylated, formylated, lipoylated, myristolylated, palmitoylated, alkylated, methylated, isoprenylated, prenylated, amidated, glycosylated, hydroxylated, iodinated, adenylated, phosphorylated, sulfated, and selenoylated, ubiquitinated. In some embodiments, the vesicle markers comprise non-proteins such as lipids, carbohydrates, nucleic acids, RNA, DNA, etc.

In several embodiments, the specific capture of vesicles based on their surface markers also enables a “dip stick” format where each different type of vesicle is captured by dipping probes coated with different capture molecules (e.g., antibodies with different specificities) into a patient sample.

Free extracellular RNA is quickly degraded by nucleases, making it a potentially poor diagnostic marker. As described above, some extracellular RNA is associated with particles or vesicles that can be found in various biological samples, such as urine. This vesicle associated RNA, which includes mRNA, is protected from the degradation processes. Microvesicles are shed from most cell types and consist of fragments of plasma membrane. Microvesicles contain RNA, mRNA, microRNA, and proteins and mirror the composition of the cell from which they are shed. Exosomes are small microvesicles secreted by a wide range of mammalian cells and are secreted under normal and pathological conditions. These vesicles contain certain proteins and RNA including mRNA and microRNA. Several embodiments evaluate nucleic acids such as small interfering RNA (siRNA), tRNA, and small activating RNA (saRNA), among others.

In several embodiments the RNA isolated from vesicles from the urine of a patient is used as a template to make complementary DNA (cDNA), for example through the use of a reverse transcriptase. In several embodiments, cDNA is amplified using the polymerase chain reaction (PCR). In other embodiments, amplification of nucleic acid and RNA may also be achieved by any suitable amplification technique such as nucleic acid based amplification (NASBA) or primer-dependent continuous amplification of nucleic acid, or ligase chain reaction. Other methods may also be used to quantify the nucleic acids, such as for example, including Northern blot analysis, RNAse protection assay, RNA sequencing, RT-PCR, real-time RT-PCR, nucleic acid sequence-based amplification, branched-DNA amplification, ELISA, mass spectrometry, CHIP-sequencing, and DNA or RNA microarray analysis.

In several embodiments, mRNA is quantified by a method entailing cDNA synthesis from mRNA and amplification of cDNA using PCR. In one preferred embodiment, a multi-well filterplate is washed with lysis buffer and wash buffer. A cDNA synthesis buffer is then added to the multi-well filterplate. The multi-well filterplate can be centrifuged. PCR primers are added to a PCR plate, and the cDNA is transferred from the multi-well filterplate to the PCR plate. The PCR plate is centrifuged, and real time PCR is commenced.

Another preferred embodiment comprises application of specific antisense primers during mRNA hybridization or during cDNA synthesis. In several embodiments, it is preferable that the primers be added during mRNA hybridization, so that excess antisense primers may be removed before cDNA synthesis to avoid carryover effects. The oligo(dT) and the specific primer (NNNN) simultaneously prime cDNA synthesis at different locations on the poly-A RNA. The specific primer (NNNN) and oligo(dT) cause the formation of cDNA during amplification. Even when the specific primer-derived cDNA is removed from the GenePlate by heating each well, the amounts of specific cDNA obtained from the heat denaturing process (for example, using TaqMan quantitative PCR) is similar to the amount obtained from an un-heated negative control. This allows the heat denaturing process to be completely eliminated. Moreover, by adding multiple antisense primers for different targets, multiple genes can be amplified from the aliquot of cDNA, and oligo(dT)-derived cDNA in the GenePlate can be stored for future use.

An additional embodiment involves a device for high-throughput quantification of mRNA from urine (or other fluids). The device includes a multi-well filterplate containing: multiple sample-delivery wells, an exosome-capturing filter (or filter directed to another biological component of interest) underneath the sample-delivery wells, and an mRNA capture zone under the filter, which contains oligo(dT)-immobilized in the wells of the mRNA capture zone. In order to increase the efficiency of exosome collection, several filtration membranes can be layered together.

In some embodiments, amplification comprises conducting real-time quantitative PCR (TaqMan) with exosome-derived RNA and control RNA. In some embodiments, a Taqman assay is employed. The 5′ to 3′ exonuclease activity of Taq polymerase is employed in a polymerase chain reaction product detection system to generate a specific detectable signal concomitantly with amplification. An oligonucleotide probe, nonextendable at the 3′ end, labeled at the 5′ end, and designed to hybridize within the target sequence, is introduced into the polymerase chain reaction assay. Annealing of the probe to one of the polymerase chain reaction product strands during the course of amplification generates a substrate suitable for exonuclease activity. During amplification, the 5′ to 3′ exonuclease activity of Taq polymerase degrades the probe into smaller fragments that can be differentiated from undegraded probe. In other embodiments, the method comprises: (a) providing to a PCR assay containing a sample, at least one labeled oligonucleotide containing a sequence complementary to a region of the target nucleic acid, wherein the labeled oligonucleotide anneals within the target nucleic acid sequence bounded by the oligonucleotide primers of step (b); (b) providing a set of oligonucleotide primers, wherein a first primer contains a sequence complementary to a region in one strand of the target nucleic acid sequence and primes the synthesis of a complementary DNA strand, and a second primer contains a sequence complementary to a region in a second strand of the target nucleic acid sequence and primes the synthesis of a complementary DNA strand; and wherein each oligonucleotide primer is selected to anneal to its complementary template upstream of any labeled oligonucleotide annealed to the same nucleic acid strand; (c) amplifying the target nucleic acid sequence employing a nucleic acid polymerase having 5′ to 3′ nuclease activity as a template dependent polymerizing agent under conditions which are permissive for PCR cycling steps of (i) annealing of primers and labeled oligonucleotide to a template nucleic acid sequence contained within the target region, and (ii) extending the primer, wherein said nucleic acid polymerase synthesizes a primer extension product while the 5′ to 3′ nuclease activity of the nucleic acid polymerase simultaneously releases labeled fragments from the annealed duplexes comprising labeled oligonucleotide and its complementary template nucleic acid sequences, thereby creating detectable labeled fragments; and (d) detecting and/or measuring the release of labeled fragments to determine the presence or absence of target sequence in the sample.

In alternative embodiments, a Taqman assay is employed that provides a reaction that results in the cleavage of single-stranded oligonucleotide probes labeled with a light-emitting label wherein the reaction is carried out in the presence of a DNA binding compound that interacts with the label to modify the light emission of the label. The method utilizes the change in light emission of the labeled probe that results from degradation of the probe. The methods are applicable in general to assays that utilize a reaction that results in cleavage of oligonucleotide probes, and in particular, to homogeneous amplification/detection assays where hybridized probe is cleaved concomitant with primer extension. A homogeneous amplification/detection assay is provided which allows the simultaneous detection of the accumulation of amplified target and the sequence-specific detection of the target sequence.

In alternative embodiments, real-time PCR formats may also be employed. One format employs an intercalating dye, such as SYBR Green. This dye provides a strong fluorescent signal on binding double-stranded DNA; this signal enables quantification of the amplified DNA. Although this format does not permit sequence-specific monitoring of amplification, it enables direct quantization of amplified DNA without any labeled probes. Other such fluorescent dyes that may also be employed are SYBR Gold, YO-PRO dyes and Yo Yo dyes.

Another real-time PCR format that may be employed uses reporter probes that hybridize to amplicons to generate a fluorescent signal. The hybridization events either separate the reporter and quencher moieties on the probes or bring them into closer proximity. The probes themselves are not degraded and the reporter fluorescent signal itself is not accumulated in the reaction. The accumulation of products during PCR is monitored by an increase in reporter fluorescent signal when probes hybridize to amplicons. Formats in this category include molecular beacons, dual-hybe probes, Sunrise or Amplifluor, and Scorpion real-time PCR assays.

Another real-time PCR format that may also be employed is the so-called “Policeman” system. In this system, the primer comprises a fluorescent moiety, such as FAM, and a quencher moiety which is capable of quenching fluorescence of the fluorescent moiety, such as TAMRA, which is covalently bound to at least one nucleotide base at the 3′ end of the primer. At the 3′ end, the primer has at least one mismatched base and thus does not complement the nucleic acid sample at that base or bases. The template nucleic acid sequence is amplified by PCR with a polymerase having 3′-5′ exonuclease activity, such as the Pfu enzyme, to produce a PCR product. The mismatched base(s) bound to the quencher moiety are cleaved from the 3′ end of the PCR product by 3′-5′ exonuclease activity. The fluorescence that results when the mismatched base with the covalently bound quencher moiety is cleaved by the polymerase, thus removing the quenching effect on the fluorescent moiety, is detected and/or quantified at least one time point during PCR. Fluorescence above background indicates the presence of the synthesized nucleic acid sample.

Another alternative embodiment involves a fully automated system for performing high throughput quantification of mRNA in biological fluid, such as urine, including: robots to apply urine samples, hypotonic buffer, and lysis buffer to the device; an automated vacuum aspirator and centrifuge, and automated PCR machinery.

The method of determining the presence of post-transplant kidney disease or condition disclosed may also employ other methods of measuring mRNA other than those described above. Other methods which may be employed include, for example, Northern blot analysis, Rnase protection, solution hybridization methods, semi-quantitative RT-PCR, and in situ hybridization.

In some embodiments, in order to properly quantify the amount of mRNA, quantification is calculated by comparing the amount of mRNA encoding a marker of disease or condition to a reference value. In some embodiments the reference value will be the amount of mRNA found in healthy non-diseased patients. In other embodiments, the reference value is the expression level of a house-keeping gene. In certain such embodiments, beta-actin, or other appropriate reference gene is used as the reference value. Numerous other house-keeping genes that are well known in the art may also be used as a reference value. In other embodiments, a house keeping gene is used as a correction factor, such that the ultimate comparison is the expression level of marker from a diseased patient as compared to the same marker from a non-diseased (control) sample. In several embodiments, the house keeping gene is a tissue specific gene or marker, such as those discussed above. In still other embodiments, the reference value is zero, such that the quantification of the markers is represented by an absolute number. In several embodiments a ratio comparing the expression of one or more markers from a diseased patient to one or more other markers from a non-diseased person is made. In several embodiments, the comparison to the reference value is performed in real-time, such that it may be possible to make a determination about the sample at an early stage in the expression analysis. For example, if a sample is processed and compared to a reference value in real time, it may be determined that the expression of the marker exceeds the reference value after only a few amplification cycles, rather than requiring a full-length analysis. In several embodiments, this early comparison is particularly valuable, such as when a rapid diagnosis and treatment plan are required (e.g., to treat heavily damaged or malfunctioning kidneys prior to kidney failure or transplant rejection).

In alternative embodiments, the ability to determine the total efficiency of a given sample by using known amounts of spiked standard RNA results from embodiments being dose-independent and sequence-independent. The use of known amounts of control RNA allows PCR measurements to be converted into the quantity of target mRNAs in the original samples.

In some embodiments, a kit is provided for extracting target components (e.g., EMV) from fluid sample, such as urine. In some embodiments, a kit comprises a capture device and additional items useful to carry out methods disclosed herein. In some embodiments, a kit comprises one or more reagents selected from the group consisting of lysis buffers, chaotropic reagents, washing buffers, alcohol, detergent, or combinations thereof. In some embodiments, kit reagents are provided individually or in storage containers. In several embodiments, kit reagents are provided ready-to-use. In some embodiments, kit reagents are provided in the form of stock solutions that are diluted before use. In some embodiments, a kit comprises plastic parts (optionally sterilized or sterilizable) that are useful to carry out methods herein disclosed. In some embodiments, a kit comprises plastic parts selected from the group consisting of racks, centrifuge tubes, vacuum manifolds, and multi-well plates. Instructions for use are also provided, in several embodiments.

In several embodiments, the analyses described herein are applicable to human patients, while in some embodiments, the methods are applicable to animals (e.g., veterinary diagnoses).

In several embodiments, presence of a post-transplant kidney condition or disease induces the altered expression of one or more markers. In several embodiments, the increased or decreased expression is measured by the amount of mRNA encoding said markers (in other embodiments, DNA or protein are used to measure expression levels). In some embodiments urine is collected from a patient and directly evaluated. In some embodiments, vesicles are concentrated, for example by use of filtration or centrifugation. Isolated vesicles are then incubated with lysis buffer to release the RNA from the vesicles, the RNA then serving as a template for cDNA which is quantified with methods such as quantitative PCR (or other appropriate amplification or quantification technique). In several embodiments, the level of specific marker RNA from patient vesicles is compared with a desired control such as, for example, RNA levels from a healthy patient population, or the RNA level from an earlier time point from the same patient or a control gene from the same patient.

### Implementation Mechanisms

According to some embodiments, the methods described herein can be implemented by one or more special-purpose computing devices. The special-purpose computing devices may be hard-wired to perform the techniques, or may include digital electronic devices such as one or more application-specific integrated circuits (ASICs) or field programmable gate arrays (FPGAs) that are persistently programmed to perform the techniques, or may include one or more general purpose hardware processors programmed to perform the techniques pursuant to program instructions in firmware, memory, other storage, or a combination. Such special-purpose computing devices may also combine custom hard-wired logic, ASICs, or FPGAs with custom programming to accomplish the techniques. The special-purpose computing devices may be desktop computer systems, server computer systems, portable computer systems, handheld devices, networking devices or any other device or combination of devices that incorporate hard-wired and/or program logic to implement the techniques.

Computing device(s) are generally controlled and coordinated by operating system software, such as iOS, Android, Chrome OS, Windows XP, Windows Vista, Windows 7, Windows 8, Windows Server, Windows CE, Unix, Linux, SunOS, Solaris, iOS, Blackberry OS, VxWorks, or other compatible operating systems. In other embodiments, the computing device may be controlled by a proprietary operating system. Conventional operating systems control and schedule computer processes for execution, perform memory management, provide file system, networking, I/O services, and provide a user interface functionality, such as a graphical user interface (“GUI”), among other things.

In some embodiments, the computer system includes a bus or other communication mechanism for communicating information, and a hardware processor, or multiple processors, coupled with the bus for processing information. Hardware processor(s) may be, for example, one or more general purpose microprocessors.

In some embodiments, the computer system may also includes a main memory, such as a random access memory (RAM), cache and/or other dynamic storage devices, coupled to a bus for storing information and instructions to be executed by a processor. Main memory also may be used for storing temporary variables or other intermediate information during execution of instructions to be executed by the processor. Such instructions, when stored in storage media accessible to the processor, render the computer system into a special-purpose machine that is customized to perform the operations specified in the instructions.

In some embodiments, the computer system further includes a read only memory (ROM) or other static storage device coupled to bus for storing static information and instructions for the processor. A storage device, such as a magnetic disk, optical disk, or USB thumb drive (Flash drive), etc., may be provided and coupled to the bus for storing information and instructions.

In some embodiments, the computer system may be coupled via a bus to a display, such as a cathode ray tube (CRT) or LCD display (or touch screen), for displaying information to a computer user. An input device, including alphanumeric and other keys, is coupled to the bus for communicating information and command selections to the processor. Another type of user input device is cursor control, such as a mouse, a trackball, or cursor direction keys for communicating direction information and command selections to the processor and for controlling cursor movement on display. This input device typically has two degrees of freedom in two axes, a first axis (e.g., x) and a second axis (e.g., y), that allows the device to specify positions in a plane. In some embodiments, the same direction information and command selections as cursor control may be implemented via receiving touches on a touch screen without a cursor.

In some embodiments, the computing system may include a user interface module to implement a GUI that may be stored in a mass storage device as executable software codes that are executed by the computing device(s). This and other modules may include, by way of example, components, such as software components, object-oriented software components, class components and task components, processes, functions, attributes, procedures, subroutines, segments of program code, drivers, firmware, microcode, circuitry, data, databases, data structures, tables, arrays, and variables.

In general, the word “module,” as used herein, refers to logic embodied in hardware or firmware, or to a collection of software instructions, possibly having entry and exit points, written in a programming language, such as, for example, Java, Lua, C or C++. A software module may be compiled and linked into an executable program, installed in a dynamic link library, or may be written in an interpreted programming language such as, for example, BASIC, Perl, or Python. It will be appreciated that software modules may be callable from other modules or from themselves, and/or may be invoked in response to detected events or interrupts. Software modules configured for execution on computing devices may be provided on a computer readable medium, such as a compact disc, digital video disc, flash drive, magnetic disc, or any other tangible medium, or as a digital download (and may be originally stored in a compressed or installable format that requires installation, decompression or decryption prior to execution). Such software code may be stored, partially or fully, on a memory device of the executing computing device, for execution by the computing device. Software instructions may be embedded in firmware, such as an EPROM. It will be further appreciated that hardware modules may be comprised of connected logic units, such as gates and flip-flops, and/or may be comprised of programmable units, such as programmable gate arrays or processors. The modules or computing device functionality described herein are preferably implemented as software modules, but may be represented in hardware or firmware. Generally, the modules described herein refer to logical modules that may be combined with other modules or divided into sub-modules despite their physical organization or storage

In some embodiments, a computer system may implement the methods described herein using customized hard-wired logic, one or more ASICs or FPGAs, firmware and/or program logic which in combination with the computer system causes or programs the computer system to be a special-purpose machine. According to one embodiment, the methods herein are performed by the computer system in response to hardware processor(s) executing one or more sequences of one or more instructions contained in main memory. Such instructions may be read into main memory from another storage medium, such as a storage device. Execution of the sequences of instructions contained in main memory causes processor(s) to perform the process steps described herein. In alternative embodiments, hard-wired circuitry may be used in place of or in combination with software instructions.

The term “non-transitory media,” and similar terms, as used herein refers to any media that store data and/or instructions that cause a machine to operate in a specific fashion. Such non-transitory media may comprise non-volatile media and/or volatile media. Non-volatile media includes, for example, optical or magnetic disks, or other types of storage devices. Volatile media includes dynamic memory, such as a main memory. Common forms of non-transitory media include, for example, a floppy disk, a flexible disk, hard disk, solid state drive, magnetic tape, or any other magnetic data storage medium, a CD-ROM, any other optical data storage medium, any physical medium with patterns of holes, a RAM, a PROM, and EPROM, a FLASH-EPROM, NVRAM, any other memory chip or cartridge, and networked versions of the same.

Non-transitory media is distinct from but may be used in conjunction with transmission media. Transmission media participates in transferring information between nontransitory media. For example, transmission media includes coaxial cables, copper wire and fiber optics, including the wires that comprise a bus. Transmission media can also take the form of acoustic or light waves, such as those generated during radio-wave and infra-red data communications.

Various forms of media may be involved in carrying one or more sequences of one or more instructions to a processor for execution. For example, the instructions may initially be carried on a magnetic disk or solid state drive of a remote computer. The remote computer can load the instructions into its dynamic memory and send the instructions over a telephone line using a modem or other network interface, such as a WAN or LAN interface. A modem local to a computer system can receive the data on the telephone line and use an infra-red transmitter to convert the data to an infra-red signal. An infra-red detector can receive the data carried in the infra-red signal and appropriate circuitry can place the data on a bus. The bus carries the data to the main memory, from which the processor retrieves and executes the instructions. The instructions received by the main memory may retrieve and execute the instructions. The instructions received by the main memory may optionally be stored on a storage device either before or after execution by the processor.

In some embodiments, the computer system may also include a communication interface coupled to a bus. The communication interface may provide a two-way data communication coupling to a network link that is connected to a local network. For example, a communication interface may be an integrated services digital network (ISDN) card, cable modem, satellite modem, or a modem to provide a data communication connection to a corresponding type of telephone line. As another example, a communication interface may be a local area network (LAN) card to provide a data communication connection to a compatible LAN (or WAN component to communicate with a WAN). Wireless links may also be implemented. In any such implementation, a communication interface sends and receives electrical, electromagnetic or optical signals that carry digital data streams representing various types of information.

A network link may typically provide data communication through one or more networks to other data devices. For example, a network link may provide a connection through a local network to a host computer or to data equipment operated by an Internet Service Provider (ISP). The ISP in turn provides data communication services through the world wide packet data communication network now commonly referred to as the “Internet.” The local network and Internet both use electrical, electromagnetic or optical signals that carry digital data streams. The signals through the various networks and the signals on the network link and through a communication interface, which carry the digital data to and from the computer system, are example forms of transmission media.

In some embodiments, the computer system can send messages and receive data, including program code, through the network(s), the network link, and the communication interface. In the Internet example, a server might transmit a requested code for an application program through the Internet, ISP, local network, and communication interface.

The received code may be executed by a processor as it is received, and/or stored in a storage device, or other non-volatile storage for later execution.

### Examples

Specific embodiments will be described with reference to the following examples which should be regarded in an illustrative rather than a restrictive sense.

### Patients and Samples

This study was reviewed and approved by the institutional review board at Sapporo City General Hospital. Kidney transplant patients (N=205) were recruited from those who received kidney transplantation at our institute. Up to 15 mL spot urine samples (N=437) were collected during the hospitalization and post-operation check up with an informed consent. The samples were stored at room temperature up to 3 hours and at −80° C. until analysis. Post-transplant complications were diagnosed based on eGFR, urinary protein and kidney biopsy with Banff criteria 2011 (Supplementary Table 1).

Urinary EMV mRNA Analysis

EMV mRNA assay was conducted as previously described. Frozen urine samples were thawed in a 37° C. water bath and centrifuged at 800×g for 15 min to remove large particles such as urinary cells and casts. Ten mL supernatants including EMV were processed by Exosome Isolation Tube (Hitachi Chemical Diagnostics, Inc. (HCD)), and followed by EMV lysis, mRNA isolation and cDNA synthesis using oligo(dT)-immobilized microplate (HCD). Sixty four mRNA were quantified by real-time PCR using ViiA7 Real-Time PCR System (Life Technologies). Those biomarker candidates were selected from those differentially expressed in kidney rejections and corresponding expression levels in urinary EMV of a healthy subject (unpublished RNA-seq data). For reference genes, GAPDH and ACTB were analyzed. Among those, ANXA1 was selected in this study. Primer sequences for ANXA1, GAPDH and ACTB were available in Table 4 below. Expression level of ANXA1 was normalized by that of GAPDH using delta Ct method with a cut off value of 6. In the delta Ct method, the ANAX1 cycle threshold (Ct) value of a sample is subtracted from the Ct value of a house-keeping gene (e.g., ACTB, GAPDH) for that same sample. Thus, the smaller the delta Ct value, the higher the ANAX1 gene expression. As shown in FIG. 3, the delta Ct value in stable recovery patients was between 5 and 6, while the delta Ct value in ABMR patients was about 2. This indicates that the delta Ct value decreased between 3 and 4 in ABMR patients, corresponding to the roughly 14-fold increase that was reported above. Statistical significance was determined by Mann-Whitney-Wilcoxon test at p value less than 1% or 5%. Data analysis was done using R (R foundation, version 3.2.0) and AUC calculation was done by ‘ROCR’ package. The sense and anti-sense primers used for the PCR analysis are presented in Table 4 below.

TABLE 4
Primer sequences used in quantitative real-time PCR analysis.

| Gene | Sense primer | Anti-sense primer |
|---|---|---|
| annexin A1 (ANXA1) | AAAGGTGGTCCCGGATCAG | TTATGCAAGGCAGCGACATC |
| glyceraldehyde-3- | CCCACTCCTCCACCTTTGAC | CATACCAGGAAATGAGCTTGACAA |
| phosphate | | |
| dehydrogenase | | |
| (GAPDH) | | |
| actin, beta (ACTB) | TTTTTCCTGGCACCCAGCACAAT | TTTTTGCCGATCCACACGGAGTACT |

### CONCLUSION

In conclusion, an innovative strategy that is safe and effective for monitoring the post-kidney transplant condition of a patient is herein disclosed. The methods of the present application can provide a promising diagnostic and prognostic assay that is non-invasive and identifies kidney transplant rejection and other complications in advance of the current standard practice (e.g., biopsy). The methods also indicate in a more targeted way than the current standard practice when a biopsy should be performed.

It is contemplated that various combinations or subcombinations of the specific features and aspects of the embodiments disclosed above may be made and still fall within one or more of the inventions. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed inventions. Thus, it is intended that the scope of the present inventions herein disclosed should not be limited by the particular disclosed embodiments described above. Moreover, while the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication. For example, actions such as “treating a subject for a disease or condition” include “instructing the administration of treatment of a subject for a disease or condition.”

Conditional language, such as, among others, “can,” “could,” “might,” or “may,” unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments.

Terms, such as, “first”, “second”, “third”, “fourth”, “fifth”, “sixth”, “seventh”, “eighth”, “ninth”, “tenth”, or “eleventh” and more, unless specifically stated otherwise, or otherwise understood within the context as used, are generally intended to refer to any order, and not necessarily to an order based on the plain meaning of the corresponding ordinal number. Therefore, terms using ordinal numbers may merely indicate separate individuals and may not necessarily mean the order therebetween. Accordingly, for example, first and second biomarkers used in this application may mean that there are merely two sets of biomarkers. In other words, there may not necessarily be any intention of order between the “first” and “second” sets of data in any aspects.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers. For example, “about 10 nanometers” includes “10 nanometers.”

What is claimed is:

1. A method of detecting the presence of a post-kidney transplant complication in a subject comprising detecting the levels of ANXA1 in a urine sample from the subject, wherein ANXA1 is in urinary exosomes and microvesicles, and wherein the detection of an elevated level of at least one marker indicates the presence of post-kidney transplant complication in the subject.

2. The method of claim 1, wherein the post-kidney transplant complication is selected from the group consisting of acute rejection, chronic rejection, borderline, interstitial fibrosis and tubular atrophy, immunoglobulin A (IgA) nephropathy and calcineurin inhibitor (CNI) toxicity.

3. The method of claim 1, wherein the post-kidney transplant complication is selected from the group consisting of acute rejection, chronic rejection, interstitial fibrosis and tubular atrophy.

4. The method of claim 1, further comprising a step to detect a reference gene selected from the group consisting of ACTB and GAPDH, wherein said reference gene is used to normalize a level of the at least one marker.

5. The method of claim 1, a said elevated level is more than 2-fold increase compared to the level of a said marker in a urine sample of a donor without post-kidney transplant complications.

6. A method for screening a human subject for an expression of an RNA associated with a post-kidney transplant complication, the method comprising comparing an expression of said RNA in a vesicle isolated from a urine sample from said subject with an expression of said RNA in a vesicle isolated from a urine sample of a donor without post-kidney transplant complications, wherein said RNA associated with a post-kidney transplant complication is ANXA1, wherein an increase in said expression of said RNA of said subject compared to said expression of said RNA of said donor indicates said subject has a post-kidney transplant complication when said increase is beyond a threshold level,
wherein said comparing said expression of said RNA in said vesicle isolated from said urine sample further comprises:
(a) capturing said vesicle from said sample from said subject by moving said sample from said subject across a vesicle-capturing filter,
(b) loading a lysis buffer onto said vesicle-capturing filter, thereby lysing said vesicle to release a vesicle-associated RNA,
(c) quantifying said expression of said RNA associated with a post-kidney transplant complication in said vesicle-associated RNA by PCR.

7. The method of claim 6, wherein quantifying said expression of said RNA by PCR comprises:
contacting said vesicle-associated RNA with a reverse transcriptase to generate complementary DNA (cDNA);
contacting said cDNA with sense and antisense primers that are specific for said RNA associated with a post-kidney transplant complication and with a DNA polymerase to generate amplified DNA;
contacting said cDNA with sense and antisense primers that are specific for a reference RNA and with said DNA polymerase to generate amplified DNA; and
using analytical software to determine an expression level or quantity or amount for said RNA.

8. The method of claim 7, wherein using analytical software to determine an expression level or quantity or amount for said RNA associated with a post-kidney transplant complication comprises:
using analytical software to determine a marker cycle threshold (Ct) value for said RNA associated with a post-kidney transplant complication;
using analytical software to determine a reference Ct value for a reference RNA; and
subtracting the marker Ct value from the reference Ct value to obtain a marker delta Ct value.

9. The method of claim 8, wherein said reference RNA is selected from the group consisting of ACTB and GAPDH.

10. The method of claim 6, wherein said increase is beyond said threshold level when said marker delta Ct value is less than 6.

11. The method of claim 6, further comprising comparing the marker delta Ct value to a control delta Ct value, the control delta Ct value being determined by subtracting a control marker Ct value from a control reference Ct value, the control marker Ct value being a Ct value of said RNA associated with a post-kidney transplant complication in urinary vesicles of a healthy donor population, the control reference Ct value being a Ct value of said reference RNA in urinary vesicles of a healthy donor population.

12. The method of claim 10, wherein said increase is beyond said threshold level when said marker delta Ct value is at least 2 less than said control delta Ct value.


SEQUENCE LISTING (6 sequences)

SEQ ID NO: 1 - DNA, 19 bp, human
aaaggtggtcccggatcag

SEQ ID NO: 2 - DNA, 20 bp, human
ttatgcaaggcagcgacatc

SEQ ID NO: 3 - DNA, 20 bp, human
cccactcctccacctttgac

SEQ ID NO: 4 - DNA, 24 bp, human
cataccaggaaatgagcttgacaa

SEQ ID NO: 5 - DNA, 23 bp, human
tttttcctggcacccagcacaat

SEQ ID NO: 6 - DNA, 25 bp, human
tttttgccgatccacacggagtact